(19) United States
(12) Patent Application Publication
(10) Pub. No.: US 2002/0144681 A1
(43) Pub. Date: Oct. 10, 2002

(54) Heating device for respiration gas

(75) Inventors: Goran Cewers, Lund (SE); Goran Rydgren, Bunkeflostrand (SE)

Correspondence Address: SCHIFF HARDIN & WAITE, 6600 SEARS TOWER, 233 S WACKER DR, CHICAGO, IL, 60606-6473, US

(73) Assignee: Siemens Elema AB

(21) Appl. No.: 10/105,786

(22) Filed: Mar. 25, 2002

(30) Foreign Application Priority Data
Apr. 4, 2001 (SE) 0101204-6

(51) Int. Cl. A61M 15/00; H05B 3/00; F24J 3/00; A62B 7/00

(52) U.S. Cl. 128/203.17; 128/203.12

(57) ABSTRACT

A heating device has a microwave source and a radiating antenna for coupling microwaves from the source into respiration gas in a gas flow-path of a patient breathing circuit, such as in an expiration gas-flow section internally located in a mechanical breathing aid. The microwave source and antenna are arranged to couple microwave energy sufficient to heat respiration gas in the flow path at a location at or upstream of a measuring device, such as a flow meter, to prevent condensation at the device. A detector and signal processor also are provided to calculate a moisture content from the microwave interaction with the respiration gas.

### BACKGROUND OF THE INVENTION

1. Field of the Invention

The present invention relates to a heating device for respiration gas and in particular to a device for heating respiration gas in an expiration gas flow path of a patient breathing system to prevent water vapor condensation therein.

2. Description of the Prior Art

In a patient breathing system for providing mechanical breathing assistance to a patient, typically separate inspiration and expiration lines are respectively connected to inspiration and expiration gas flow sections of a mechanical breathing aid and to a common gas flow conduit, such as an endotracheal tube. The endotracheal tube is connectable with a patient's airways to form inspiration and expiration gas flowpaths for respiration gas passing to and from the airways. Respiration gas flowing into the inspiration line from the breathing aid often has water vapor added from a humidifier in order to maintain adequate moisture levels within the patient. Respiration gas expired by a patient naturally contains water vapor. It is important to avoid, or at least control, the condensation of the water vapor within gas flowpaths of the patient breathing system.

Water vapor condensate, particularly within the inspiration gas flowpath, is potentially hazardous since as it pools it may restrict the flow of respiration gas to the patient or the pooled water may be inhaled by the patient.

Gas expired by the patient is generally saturated with water vapor and also has a relatively high temperature, about 36° C. Ambient temperature around the patient where the measurement equipment and the expiration line is located is usually around 18-22° C. Cooling of expired air thus is generally unavoidable and condensation therefore forms.

Various measurement instruments are often provided in fluid connection with, particularly, the expiration gas flowpath. Many flow meters operate less effectively, and may even produce erroneous measurements, if water vapor in the flowing respiration gas condenses within the meter. Other measurement instruments, such as various gas analyzers, are also sensitive to condensation inside the measurement instrument. Preventing condensation of water vapor within the measurement instrument, particularly the flow meter, is especially important in the supply of mechanical breathing assistance to a patients, since the function of the breathing aid relies on the correct operation of the measurement instrument.

Moreover it is useful to have a measure of the amount of water vapor that is present in the respiration gas. For example, the respective flows of the respiration gas in the inspiration gas flowpath and in the expiration gas flowpath are often compared in order to monitor for leakage. Since water vapor is present in an unknown amount in respiration gas expired by a patient, the volume of expired gas measured by a flow meter within the expiration gas flowpath is increased by an unknown amount because of this water vapor. This makes such a comparison unreliable unless the amount of water vapor in the gas expired by the patient is measured.

It is known, for example, from U.S. Pat. No. 5,988,164 to provide a heating means in the form of an electrical heating wire or a liquid filled heating tube located within one or both the inspiration line and the expiration line of a patient breathing system in order to prevent condensation within the associated line or lines. A separate solid state sensor of a known type is provided for measuring the relative humidity of (the amount of water vapor present in) the respiration gas in the line or lines being heated.

### SUMMARY OF THE INVENTION

It is an object of the present invention to provide a heating device suitable for use in a patient breathing system, and a patient breathing system employing a heating device, to alleviate water vapor condensation in gas flowpaths of the breathing system and which allows a measurement of the amount of water vapor in the respiration gas to be measured.

The above object is achieved in accordance with the principles of the present invention in a heating device and in a patient breathing system wherein a microwave source is provided for generating microwave energy at a power to elevate the temperature of respiration gas sufficiently to inhibit water vapor condensation from the respiration gas, and a microwave energy coupler for coupling the microwave energy from the source into a gas flow channel in which the respiration gas flows.

By directing microwave radiation, preferably at only one or more of those wavelengths preferentially absorbed by water vapor, of sufficient power, typically between 0.5 Watts (W) and 10 W, into the respiration gas within one or both of the inspiration and the expiration gas flowpaths, both a warming and a measure of the amount of water vapor present can be achieved without significantly affecting the gas flow.

An antenna may be employed to couple microwaves into the respiration gas and the antenna coupling factor measured using known circuitry as an indication of the amount of water vapor present in the gas. Thus the intensity of microwave energy after transmission through the respiration gas need not be measured. This has an advantage that a lower intensity can be produced by the microwave source.

### DESCRIPTION OF THE DRAWINGS

FIG. 1 is a schematic representation of a first embodiment according to the present invention within a patient breathing system.

FIG. 1A is an enlarged detail from FIG. 1.

FIG. 2 is a schematic representation of a second embodiment according to the present invention.

### DESCRIPTION OF THE PREFERRED EMBODIMENTS

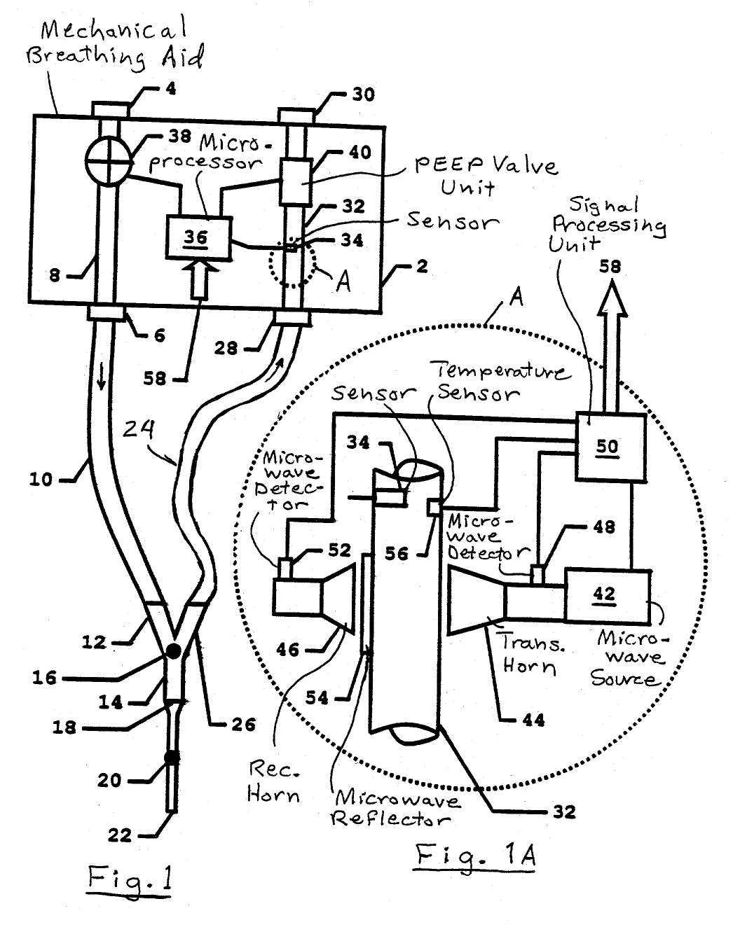

As shown in FIG. 1., a mechanical breathing aid 2, such as a ventilator or anaesthetic device, has an inspiration gas inlet 4 connected to an inspiration gas outlet 6 via an inspiration gas flow section 8. The outlet 6 is connected to supply respiration gas from the breathing aid 2 into an inspiration line 10 which is connected via a first limb 12 and a common flow limb 14 of a Y-piece 16 to a first end 18 of an endotracheal tube 20. The endotracheal tube 20 has an open end 22 for communicating with the airways of a patient (not shown). The inspiration gas flow section 8, the inspiration line 10 and the endotracheal tube 20 thus form an inspiration gas flow-path for respiration gas to the patient's airways.

The endotracheal tube 20 also is connected via the Y-piece 16 to an expiration line 24 which is connected between a second limb 26 of the Y-piece 10 and an expiration gas inlet 28 of the breathing aid 2. An expiration gas outlet 30 is provided in the breathing aid 2 connected to the expiration gas inlet 28 via an expiration gas flow section 32. The expiration gas flow section 32, the expiration line 24 and the endotracheal tube 20 thus form an expiration gas flowpath for respiration gas from the patient's airways.

A measurement instrument, here a flow sensor 34, is located in the expiration gas flow-path, here shown as being located within the expiration gas flow section 32 of the mechanical breathing aid 2, to measure flow of respiration gas from the patient. A microprocessor control unit 36 is operably connected to the sensor 34 to receive a signal therefrom indicative of the magnitude of the sensed flow with which the microprocessor 36 may calculate the volume of respiration gas flowing from the patient in a known manner. This may then be used in a known manner as a control parameter in the operation of the inspiration gas flow regulatory system, depicted as an automatically actuated flow control valve unit 38 connected to the inspiration gas flow section 8, and/or the expiration gas flow regulatory system, here depicted as an automatically controlled PEEP-valve unit 40. It will be appreciated by those skilled in the art that the general operation of the breathing aid 2 in controlling the flow of respiratory gas within the inspiration and the expiration gas flow-paths is well known and so will be described only in sufficient detail as to permit an understanding of the present invention.

FIG. 1A shows an enlarged detail A of a portion of the expiration gas flow-path, here a portion of the expiration gas flow section 32, within the breathing aid 2. A heating device according to the present invention has a microwave source 42, such as a klystron, magnetron or solid state microwave device, capable of providing a power output of up to about 10W which is sufficient to warm respiration gas flowing past the flow sensor 34 and inhibit condensation at that location. Preferably the source 42 delivers microwaves at a frequency or frequencies preferentially absorbed by water vapor, such as at 20 GHz or 2.45 GHz employed in domestic microwave ovens. The source 42 has an output connected to a transmitting horn antenna 44 which, in the present example, is disposed relative to the expiration gas flow section 32 so as to be able to couple microwave energy from the source 42 into respiration gas within the section 32 at a location proximal that of the flow meter 34 to warm passing respiration gas. Furthermore, the flow sensor 34 or other measurement instrument may be located within other parts of the gas flow-paths, such as within the expiration line 24 or inspiration line 10, in which case the heating device as described herein will be located to warm respiration gas within the relevant portion of the gas flow paths.

A receiving horn antenna 46 is disposed so as to be able to collect transmitted microwave energy after passage through respiration gas within the expiration gas flowpath. In the present embodiment since both horn antennas 44,46 are located outside the expiration gas flow section 32, the flow of respiration gas within the gas flowpath will be substantially unaffected by the exemplary heating device of the present invention. A microwave detector 48 is connected to the transmitting horn 44 to monitor the intensity of the microwaves being transmitted into the expiration gas flow section 32 and has an output connected to a signal processing unit 50. A similar microwave detector 52 is connected to the receiving horn 46 to monitor the intensity of the microwaves after their interaction with respiration gas in the expiration gas flow section 32 and also has an output connected to the signal processing unit 50. It will be appreciated that, as an alternative, a microwave reflector element 54 may replace the receiving horn 46 and be arranged to reflect transmitted microwave energy either back to the transmission horn 44 where the detector 48 may be employed to also measure the intensity of reflected microwave energy after its transmission through the respiration gas in a manner known in the art or to a receiving horn (not shown) located on the same side of the expiration gas flow section 32 as the transmitting horn 44. This reflector element 54 might conveniently be a metallic mesh located within the gas flow section 32, against an internal wall.

The signal processing unit 50 may be realized in analog circuitry or in digital circuitry using a programmed microprocessor which can form an integral part of the microprocessor control unit 36 connected to the flow meter 34. The signal processing unit 50 is, in the present embodiment, specifically configured to calculate an amount of microwave energy the gas has absorbed as a measure of the moisture content of the gas. In the present embodiment this amount is determined from the difference between the outputs of the detectors 48,52. Alternatively, detector 48 may be omitted and the microwave source 42 configured to generate a fixed, known intensity of microwaves, the value of which is then pre-programmed in to the signal processing unit 50 as a substitute for the intensity value obtained from the detector 48. The signal processing system 50 may be provided with an experimentally determined look-up table which equates the amount of microwave energy absorbed with moisture content and which can be constructed from calibration measurements using gas flows with known moisture content. Alternatively the signal processing system may be programmed with an empirical formula equating the amount of microwave energy absorbed with moisture content and derived from similar calibration measurements. The system 50 then accesses the look-up table or formula and, using the calculated amount of absorbed microwave energy, determines the moisture content of the gas.

In an alternative heating device, also illustrated in FIG. 1, the microwave source 42 maybe provided with a controllable power level output which is responsive to a control signal generated by the signal processing unit 50 in dependence of the amount of absorbed microwave energy. The source 42 and unit 50 are configured to cooperate to maintain the power at a level which tends to maximize the percentage of transmitted radiation which is absorbed whilst ensuring a detectable level of transmitted radiation at the detector 52.

Additionally, a temperature sensor 56 may be provided in good thermal contact with respiration gas within the expiration gas flow section 32 at a location suitable for measuring the temperature of the respiration gas warmed by the microwaves. The temperature sensor 56 provides an output indicative of this measured temperature to the signal processing unit 50 which is additionally programmed with a desired temperature value and provides the control signal to the microwave source 42 additionally dependent on the difference between these temperatures. The microwave source 42 is responsive to this control signal to control the intensity of the generated microwaves so as to tend to achieve the desired temperature of respiration gas, selected to inhibit condensation of water vapor at the flow meter 34.

The signal processing unit 50 is configured to provide an output signal 58 indicative of the determined moisture content to the microprocessor control unit 36 where it may be used, in a manner known in the art to compensate the gas volume, as determined from the flow meter 34 reading, for the presence of moisture. It will be appreciated that the determination of moisture content may be undertaken in the microprocessor control unit 36 in a manner identical to that described above with respect to the signal processing unit 50. In this case the output signal 58 from the signal processing unit 50 need only indicative of the difference value or indeed only the output signals from the two microwave detectors 48,52. It will be further appreciated that, as mentioned above, the functionality of the signal processing unit 50 may be readily programmed in to the microprocessor control unit 36.

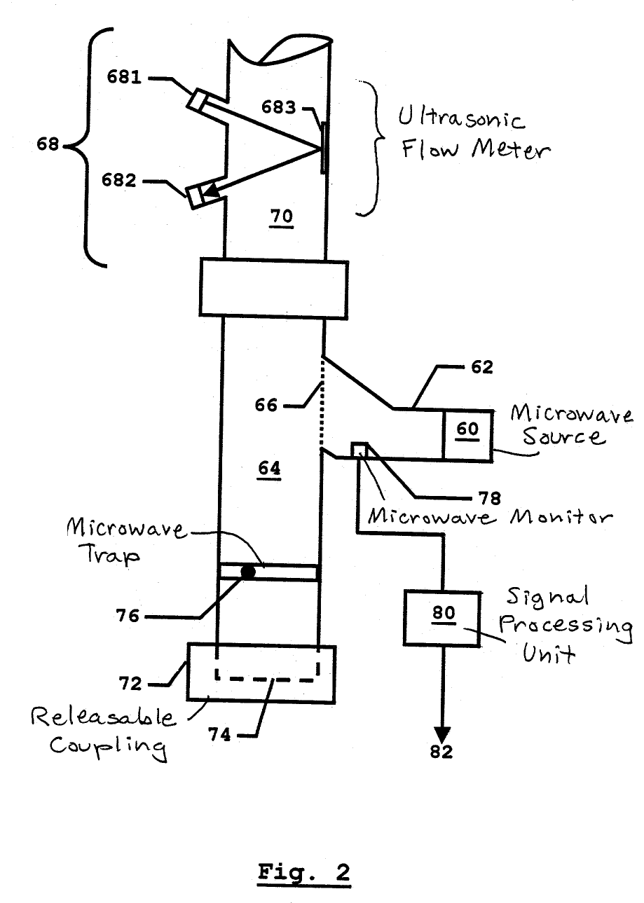

A further heating device according to the present invention is shown in FIG. 2. This device has a microwave source 60 coupled to supply microwave energy to one end of a waveguide antenna element 62. The waveguide antenna element 62 is configured to transmit supplied microwave energy into a gas flow channel 64, through a microwave transparent, gas tight, wall section 66, at an angle in a direction toward an ultrasonic flow meter 68 of know construction and operation. The flow meter 68, illustrated in FIG. 2, comprises an ultrasound emitter 681, an ultrasound receiver 682, and an ultrasound reflection element 683 (which may be integrated with a gas flow channel 70) and a transit time of an ultrasound signal which is passed from the transmitter 681 to the receiver 682 as indicated by the arrow is used to determine the flow magnitude.

The gas flow channel 64 of the heating device may be releasably connectable with the gas flow channel 70 of the ultrasonic flow meter 68, as shown in the FIG. 2, or maybe formed integrated with it and provided with a releasable coupling 72 at an open end 74 for in-line connection to an inspiration or an expiration gas flow-path for respiration gas to or from a patient, such as are shown in FIG. 1. A microwave trap 76 is preferably arranged within the gas flow channel 64 of the heating device when the device is intended for connection in to an expiration gas flow-path with the open end 74 directed toward a patient. Such a trap 76 may have a number of long thin plates arranged to provide channels extending in a direction substantially parallel to the direction of gas flow. If a trap 76 is employed, a visible indication (such as an arrow indicating which end 74 should be located toward a patient) of the intended orientation of the device should be provided on an externally visible surface of the channel 64.

The heating device further has a microwave monitor 78, located within the waveguide element 62 which provides an output representative of the level of microwave power directed towards the transparent wall section 66 as well as the level of power reflected back from gas proximal the wall section 66. The intensity of reflected microwaves will be dependent on the level of moisture within respiration gas flowing in the flow channel 64 as this will effect the coupling of microwaves in to the gas. This monitor may be readily constructed using known circuitry employed in known microwave power-meters and includes a suitably disposed directional coupler which is rotatable to selectively couple microwaves passing in one of two opposing directions (that is in directions towards or back from the wall section 66) and is operably connected to a microwave detector to detect the level of coupled microwaves.

A signal processing unit 80 receives the outputs from the monitor 78 indicative of the intensities of microwaves which are travelling in opposite directions in the waveguide 62 and in a known manner determines a microwave coupling factor dependent on the supplied and reflected microwave power. Since the reflection of microwaves from the proximal the transparent wall section 66 is dependent on the moisture content of the respiration gas then the microwave coupling factor provides a measure of this moisture content.

As with the signal processing unit 50 of FIG. 1 the signal processing unit 80 may be provided with look-up tables or an empirically determined formula to enable a determination of moisture content to be made from the so measured coupling factor. A signal representative of the so determined moisture content can be provided as an output 82 from the signal processing unit 80 which may be employed in the manners described with respect to the output 58 of the signal processing unit 50 of FIG. 1.

Although the heating device according to the present invention is exemplified by FIG. 1 and FIG. 2 as being located proximal a flow sensor it will be appreciated that the application of the heating device of the present invention is not limited to this placement but rather may be located within the flow path for respiration gas as required.

Although modifications and changes maybe suggested by those skilled in the art, it is in the intention of the inventors to embody within the patent warranted hereon all changes and modifications as reasonably and properly come within the scope of their contribution to the art.

We claim as our invention:

1. A heating device for respiration gas, containing water vapor, in a patient breathing circuit having a respiration gas flow channel, said heating device comprising:
a microwave source which generates microwave energy at a power sufficient to elevate a temperature of said respiration gas to inhibit condensation of said water vapor from said respiration gas; and
a microwave coupler adapted to couple said microwave energy generated by said microwave source into said respiration gas flow channel.

2. A heating device as claimed in claim 1 further comprising a detector adapted to be disposed in said respiration gas flow channel for generating a detector output indicative of interaction between said microwave energy and said respiration gas in said respiration gas flow channel.

3. A heating device as claimed in claim 2 wherein said microwave source has a control input connected to said detector to receive said detector output therefrom and to vary said power of said microwave energy dependent on said detector output.

4. A heating device as claimed in claim 2 further comprising a signal processor connected to said detector for receiving said detector output therefrom, said signal processor determining, from said detector output, an amount of said water vapor in said respiration gas flow channel.

5. A heating device as claimed in claim 4 wherein said detector comprises a microwave receiver for receiving microwave energy from said microwave coupler after transmission of said microwave energy through said respiration gas, and a microwave detector connected to said microwave receiver for generating, as said detector output, a signal indicating an intensity of the microwave energy received by said microwave receiver, and wherein said signal processor calculates, from said intensity, an amount of microwave energy absorbed by said respiration gas in said respiration gas flow channel.

6. A heating device as claimed in claim 5 wherein said detector further comprises a microwave monitor for monitoring an intensity of microwave energy generated by said microwave source and which generates a microwave monitor output signal indicative of said intensity of the microwave energy generated by said microwave source, and wherein said signal processor is connected to said microwave monitor and receives said microwave monitor output therefrom, and wherein said signal processor subtracts said microwave detector output from said microwave monitor output to determine said amount of microwave energy absorbed by said respiration gas in said respiration gas flow channel.

7. A heating device as claimed in claim 4 wherein said detector comprises a coupling factor measurement unit for measuring a coupling factor associated with the coupling of said microwave energy into said respiration gas, said coupling factor measurement unit generating a coupling factor signal, and said signal processor being connected to said coupling factor measurement unit for receiving said coupling factor signal therefrom.

8. A heating device as claimed in claim 1 wherein said microwave source generates said microwave energy only at frequencies preferentially absorbed by said water vapor.

9. A patient breathing device comprising:
an inspiration line;
an expiration line;
a breathing aid having an inspiration gas flow section and an expiration gas flow section respectively coupled to said inspiration line and said expiration line;
a patient section connected at a first end to each of said inspiration line and said expiration line, and having a second end adapted for connection to airways of a patient, said inspiration line, said inspiration section and said patient section forming, in combination, an inspiration gas flow path, and said expiration line, said expiration gas flow section and said patient section forming, in combination, an expiration gas flow path; and
a heating device for elevating a temperature of gas within a respiration gas flow path comprising at least one of said inspiration gas flow path and said expiration gas flow path, said heating device comprising a microwave source for generating microwave energy at a power sufficient to elevate a temperature of respiration gas in said respiration gas flow path to inhibit condensation of water vapor from said respiration gas, and a microwave coupler for coupling said microwave energy into said respiration gas flow path.

10. A patient breathing system as claimed in claim 9 further comprising a measurement device for measuring, at a measurement location, at least one property of said respiration gas in said respiration gas flow path, and wherein said heating device is disposed to elevate the temperature of said respiration gas at a location proximal to said measurement location.

11. A patient breathing system as claimed in claim 9 further comprising a detector adapted to be disposed in said respiration gas flow path for generating a detector output indicative of interaction between said microwave energy and said respiration gas in said respiration gas flow path.

12. A patient breathing system as claimed in claim 11 wherein said microwave source has a control input connected to said detector to receive said detector output therefrom and to vary said power of said microwave energy dependent on said detector output.

13. A patient breathing system as claimed in claim 11 further comprising a signal processor connected to said detector for receiving said detector output therefrom, said signal processor determining, from said detector output, an amount of said water vapor in said respiration gas flow path.

14. A patient breathing system as claimed in claim 13 wherein said detector comprises a microwave receiver for receiving microwave energy from said microwave coupler after transmission of said microwave energy through said respiration gas, and a microwave detector connected to said microwave receiver for generating, as said detector output, a signal indicating an intensity of the microwave energy received by said microwave receiver, and wherein said signal processor calculates, from said intensity, an amount of microwave energy absorbed by said respiration gas in said respiration gas flow path.

15. A patient breathing system as claimed in claim 14 wherein said detector further comprises a microwave monitor for monitoring an intensity of microwave energy generated by said microwave source and which generates a microwave monitor output signal indicative of said intensity of the microwave energy generated by said microwave source, and wherein said signal processor is connected to said microwave monitor and receives said microwave monitor output therefrom, and wherein said signal processor subtracts said microwave detector output from said microwave monitor output to determine said amount of microwave energy absorbed by said respiration gas in said respiration gas flow path.

16. A patient breathing system as claimed in claim 13 wherein said detector comprises a coupling factor measurement unit for measuring a coupling factor associated with the coupling of said microwave energy into said respiration gas, said coupling factor measurement unit generating a coupling factor signal, and said signal processor being connected to said coupling factor measurement unit for receiving said coupling factor signal therefrom.

17. A patient breathing system as claimed in claim 9 wherein said microwave source generates said microwave energy only at frequencies preferentially absorbed by said water vapor.
